(19) United States
(12) Patent Application Publication
(10) Pub. No.: US 2007/0292470 A1
(43) Pub. Date: Dec. 20, 2007

(54) Implantable Medical Devices and Methods for Making the Same

(75) Inventor: Ronan Thornton, Corcullen (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/424,303

(22) Filed: Jun. 15, 2006

(51) Int. Cl. A61F 2/02; B27N 3/00

(52) U.S. Cl. 424/423; 264/109

(57) ABSTRACT

Disclosed herein are polymeric implantable medical devices and methods for making the same. Specifically, disclosed are polymeric implantable medical devices produced through the use of solvent casting methods.

### FIELD OF THE INVENTION

The present invention relates to polymeric implantable medical devices and methods for making the same. Specifically, the present invention relates to using a solvent casting method to produce polymeric implantable medical devices including stents.

### BACKGROUND OF THE INVENTION

Coating the surface of implanted medical devices with polymers and/or bioactive materials has become a common practice. Including bioactive materials on the surface of implantable medical devices can enhance the intended effect of the medical device, reduce or eliminate infection or inflammation related to the device, accelerate or improve acceptance of the device by the body, and/or treat specific diseases at the site of the device.

One challenge in the field of implantable medical devices has been adhering bioactive materials to the surfaces of implantable devices so that the bioactive materials will be released once the device is implanted. Conventionally, coatings have been applied to medical devices by processes such as dipping or spraying. These coating processes, however, are inefficient, indiscriminate, wasteful, difficult to control, and/or are limited in the types of coating materials that they may apply. For example, because dip-coating or spray-coating processes often indiscriminately coat the internal surface of a patterned medical device as well as the external surface, expensive coating materials, such as bioactive materials, are wasted, resulting in large amounts of the coating being lost during the process. Coating efficiencies of about 4% are typically obtained with spraying techniques for the application of non-biologic therapeutic agents. While this may be tolerated for low cost coatings, such waste is prohibitive for expensive materials such as DNA (which may cost roughly $250 per mg), proteins or viruses.

Another approach to coating implantable medical devices with bioactive materials has been to include the bioactive materials in polymeric coatings. Polymeric coatings can hold bioactive materials onto the surface of implantable medical devices and release the bioactive materials via degradation of the polymer or diffusion into liquid or tissue (in this case the polymer is non-degradable).

While polymeric coatings can be used to adhere bioactive materials to implanted medical devices, there are a number of problems associated with their use as coatings on medical devices. For example, adherence of a polymeric coating to a substantially different substrate, such as a stent's metallic substrate, is difficult due to differing characteristics of the materials (such as differing thermal expansion properties). The difficulty in adhering the two different material types often leads to inadequate bonding between the medical device and the overlying polymeric coating which can result in the separation of the materials over time. Such separation is an exceptionally undesirable property in an implanted medical device. Another drawback of coating implantable medical devices with polymers is that it is difficult to evenly coat a medical device with a polymeric coating. The uneven coating of a medical device can lead to unequal drug delivery across different portions of the device. This drawback is especially apparent in relation to small implantable medical devices, such as stents.

In light of these drawbacks of coating polymers onto implantable medical devices, methods of creating stents from polymeric films have been developed. In these approaches, a polymer containing a bioactive material is formed into thin films which are then structured into implantable medical devices. See, for example, United States Patent Number (USPN) U.S. Pat. No. 6,641,831 and United States Patent Publication No. 2005/0021131. While these approaches addressed certain drawbacks of coating a stent with a polymer coating, shaping pre-made films into complex geometric patterns also has inherent difficulties and drawbacks.

Others have also attempted to create implantable medical devices comprising polymers containing bioactive materials through thermal injection molding. However, the temperatures required for polymers to undergo thermal injection molding are relatively high and above the temperature at which most bioactive materials remain stable. Therefore, this approach has also not adequately addressed the issue of providing an implantable medical device constructed of a polymer containing bioactive materials. The present invention provides such implantable medical devices and methods of making the same.

### SUMMARY OF THE INVENTION

As stated, polymers and coatings such as phosphorycholine, hydrogels and hydroxyapatite, with and without additional therapeutic agents, are commonly placed onto the surface of medical devices at the point of manufacture. Injection molding a polymeric stent can provide a desirable manufacturing method. However the temperature required to melt a polymer degrades many bioactive materials. The present invention provides a method to manufacture polymeric implantable medical devices that involve dissolving a polymer and a bioactive material in an appropriate volatile co-solvent, and then injecting the mixture into a mold ‘cold’ (i.e. at a temperature that does not degrade bioactive materials). The volatile co-solvent can then be removed from the mixture through evaporation which can be aided by, without limitation, appropriate venting, vacuuming or low level heating. In accordance with these processes, mixture viscosity can be easily tuned by adding more or less solvent. Further, the polymer can be bioresorbable or non-resorbable.

Specifically, the present invention comprises methods and medical devices made using the methods of the present invention. In one embodiment of the methods according to the present invention the method comprises forming an implantable medical device by dissolving a polymer and a bioactive material in a co-solvent to form a mixture; injecting the mixture into a mold; allowing the co-solvent to evaporate from the mixture while the mixture is in the mold; and removing the mixture from the mold after the evaporation. In another embodiment of the methods, the mold is part of a system comprising at least one evacuation port. When an evaporation port is included, evaporation can occur passively through the port or the rate of evaporation of the co-solvent can be accelerated through a method selected from the group consisting of opening one or more evaporation ports; applying a vacuum to said one or more evaporation ports; heating the system to a temperature below that which would degrade the bioactive material; and combinations thereof.

In an embodiment of the medical devices according to the present invention, the medical device comprises a polymer and a bioactive material wherein at one point the polymer and bioactive material were dissolved together in a co-solvent and injected into a mold wherein the temperature of the co-solvent was kept below a temperature at which the bioactive material would degrade. In another embodiment of the implantable medical devices, the mold was part of a system comprising at least one evaporation port and the co-solvent was allowed to evaporate from the mold. In certain embodiments of the implantable medical devices, the rate of evaporation was accelerated through a method selected from the group consisting of opening one or more evaporation ports; applying a vacuum to the one or more evaporation ports; heating the system to a temperature below that which would degrade the bioactive material; and combinations thereof.

The following descriptions of further embodiments can be applied to both the methods and the medical devices of the present invention. In certain embodiments after removal from the mold, the mixture can be further dried. The drying can occur in at least one device selected from the group consisting of an oven, a vacuum oven, a vacuum chamber, a fume hood and a laminar flow hood.

In other embodiments according to the present invention, the polymer is selected from the group consisting of polyesters, polyacrylamides, polyvinylpyrrolidone, polymethylmethacrylate, polybutylmethacrylate, polyvinyl acetate, poly-lactic acid (PLA), poly-glycolic acid (PGA), polycarbonates, polyurethanes, polycapralactone, polyorthoester and copolymers thereof.

In further embodiments, the bioactive material is selected from the group consisting of Zotarolimus (ABT-578), rapamycin, paclitaxel, dexamethasone, everolimus, tacrolimus, des-aspartate angiotensin I, exochelins, nitric oxide, apocynin, gamma-tocopheryl, pleiotrophin, estradiol, heparin, aspirin and HMG-CoA reductase inhibitors such as atorvastatin, cerivastatin, fluvastatin, lovastatin, pravastatin, rosuvastatin, simvastatin, abciximab, angiopeptin, colchicines, eptifibatide, hirudin, methotrexate, streptokinase, taxol, ticlopidine, tissue plasminogen activator, trapidil, urokinase, vascular endothelial growth factor, transforming growth factor beta, insulin growth factor, platelet-derived growth factor, fibroblast growth factor, and combinations thereof.

Co-solvents used in accordance with embodiments of the present invention can be selected from the group consisting of dimethylsulfoxide, iso-propyl alcohol, methanol, ethanol, dimethylformamide, benzene, toluene, xylene, cyclohexane, heptane, chloroform, acetone, methylene chloride, ethyl acetate, tetrahydrofuran (THF) and combinations thereof.

Certain embodiments of the methods according to the present invention are used to produce stents. Stents also comprise one embodiment of the medical devices of the present invention.

### Definition of Terms

Prior to setting forth embodiments according to the present invention, it may be helpful to an understanding thereof to set forth definitions of certain terms that will be used hereinafter. Some terms that are used herein are further described as follows:

The term “bioactive material(s)” refers to any organic, inorganic, or living agent that is biologically active or relevant. For example, a bioactive material can be a protein, a polypeptide, a polysaccharide (e.g. heparin), an oligosaccharide, a mono- or disaccharide, an organic compound, an organometallic compound, or an inorganic compound. It can include a living or senescent cell, bacterium, virus, or part thereof. It can include a biologically active molecule such as a hormone, a growth factor, a growth factor producing virus, a growth factor inhibitor, a growth factor receptor, an anti-inflammatory agent, an antimetabolite, an integrin blocker, or a complete or partial functional insense or antisense gene. It can also include a man-made particle or material, which carries a biologically relevant or active material. An example is a nanoparticle comprising a core with a drug and a coating on the core.

Bioactive materials also can include drugs such as chemical or biological compounds that can have a therapeutic effect on a biological organism. Bioactive materials include those that are especially useful for long-term therapy such as hormonal treatment. Examples include drugs for contraception and hormone replacement therapy, and for the treatment of diseases such as osteoporosis, cancer, epilepsy, Parkinson's disease and pain. Suitable biological materials can include, e.g., anti-inflammatory agents, anti-infective agents (e.g., antibiotics and antiviral agents), analgesics and analgesic combinations, antiasthmatic agents, anticonvulsants, antidepressants, antidiabetic agents, antineoplastics, anticancer agents, antipsychotics, and agents used for cardiovascular diseases such as anti-restenosis and anti-coagulant compounds. Exemplary drugs include, but are not limited to, Zotarolimus (ABT-578), rapamycin, paclitaxel, dexamethasone, everolimus, tacrolimus, des-aspartate angiotensin I, exochelins, nitric oxide, apocynin, gamma-tocopheryl, pleiotrophin, estradiol, heparin, aspirin, atorvastatin, cerivastatin, fluvastatin, lovastatin, pravastatin, rosuvastatin, simvastatin, abciximab, angiopeptin, colchicines, eptifibatide, hirudin, methotrexate, streptokinase, taxol, ticlopidine, tissue plasminogen activator, trapidil, urokinase, vascular endothelial growth factor, transforming growth factor beta, insulin growth factor, platelet-derived growth factor, fibroblast growth factor, and combinations thereof.

Bioactive materials also can include precursor materials that exhibit the relevant biological activity after being metabolized, broken-down (e.g. cleaving molecular components), or otherwise processed and modified within the body. These can include such precursor materials that might otherwise be considered relatively biologically inert or otherwise not effective for a particular result related to the medical condition to be treated prior to such modification.

Combinations, blends, or other preparations of any of the foregoing examples can be made and still be considered bioactive materials within the intended meaning herein. Aspects of the present invention directed toward bioactive materials can include any or all of the foregoing examples.

The term “medical device” refers to an entity not produced in nature, which performs a function inside or on the surface of the human body. Medical devices include but are not limited to: biomaterials, drug delivery apparatuses, catheters, vascular conduits, stents, plates, screws, spinal cages, dental implants, dental fillings, braces, artificial joints, embolic devices, ventricular assist devices, artificial hearts, heart valves, venous filters, staples, clips, sutures, prosthetic meshes, pacemakers, pacemaker leads, defibrillators, neurostimulators, neurostimulator leads, and implantable or external sensors. Medical devices are not limited by size and can include microsystems and nanosystems (wherein these systems can include, without limitation, mechanical and/or electrical systems) which perform a function in or on the surface of a human or other animal body. Embodiments of the invention include such medical devices.

The terms “implants” or “implantable” refers to a category of medical devices, which are implanted in a patient for some period of time. They can be diagnostic or therapeutic in nature, and long or short term.

The term “stents” refers to devices that are used to maintain patency of a body lumen or interstitial tract. Stents are currently used in peripheral, coronary, and cerebrovascular vessels, the alimentary, hepatobiliary, and urologic systems, the liver parenchyma (e.g., porto-systemic shunts), and the spine (e.g., fusion cages). In the future, stents will be used in smaller vessels (currently minimum stent diameters are limited to about 2 millimeters). For example, they will be used in the interstitium to create conduits between the ventricles of the heart and coronary arteries, or between coronary arteries and coronary veins. In the eye, stents are being developed for the Canal of Schlem to treat glaucoma.

### DETAILED DESCRIPTION

Implantable medical devices comprising polymers and bioactive materials have been created using solvent casting to create thin films which are then structured into appropriate shapes. See, for example, United States patent Number (USPN) U.S. Pat. No. 6,641,831 and United States Patent Application No. 2005/0021131. While these approaches addressed certain drawbacks of the prior art, shaping pre-made films into complex geometric patterns, such as those found in stents, has inherent technical difficulties and drawbacks.

Others have also attempted to create implantable medical devices comprising polymers containing bioactive materials through thermal injection molding. However, the temperatures required for polymers to undergo this process are relatively high and above the temperature at which most bioactive materials remain stable. Therefore, this approach also did not adequately address the issue of providing an implantable medical device constructed of a polymer containing bioactive materials. The present invention provides such implantable medical devices and methods for making the same.

The present invention provides methods to manufacture polymeric implantable medical devices containing bioactive materials. The methods involve dissolving a polymer and a bioactive material in an appropriate volatile co-solvent, and injecting the mixture into a mold ‘cold’ (i.e. at a temperature that does not degrade bioactive materials). The volatile co-solvent can then be removed from the mixture through evaporation. In one embodiment, evaporation can occur through a parting line in the mold. In other embodiments, evaporation can be aided by, without limitation, appropriate venting, vacuuming or low level heating. The mixture viscosity can be easily tuned by adding more or less solvent. Further, the polymer can be bioresorbable or non-resorbable. These methods can provide cost-effective means to manufacture a polymeric drug-eluting stent. Further, the methods are rapid, provide a finished stent in its final shape and can provide any surface texturing that is required. The methods can also facilitate the inclusion of three dimensional topography of a stent and can reduce bioactive materials waste by utilizing 100% of the bioactive material in the mixture.

In one example according to the present invention, instead of melting a polymer using heat, the polymer and a bioactive material are dissolved in a suitable co-solvent. The amount of co-solvent is selected to give an appropriate viscosity to the mixture. In the described example this mixture is then injected into a mold. Following injection into the mold, the co-solvent is allowed to evaporate sufficiently for the mixture (now a shaped implantable medical device) to be removed from the mold without damage or deformation. Co-solvent evaporation can be aided by, without limitation, the opening of evaporation ports in the mold, by the application of a vacuum to evaporation ports and/or by low level heating. Once the implantable medical device has been removed from the mold after sufficient co-solvent evaporation, in certain embodiments evaporation and drying can be further aided by, without limitation, an oven or other appropriate fume hood or chamber.

The following provides non-limiting exemplary polymers, bioactive materials and co-solvents that are especially beneficial for use in accordance with the present invention. Polymers: poly-lactic acid (PLA); poly-glycolic acid (PGA), polycarbonates, polyurethanes, polycapralactone and polyorthoester. Bioactive Materials: Zotarolimus (ABT-578), rapamycin, paclitaxel, dexamethasone, everolimus, tacrolimus, des-aspartate angiotensin I, exochelins, nitric oxide, apocynin, gamma-tocopheryl, pleiotrophin, estradiol, heparin, aspirin and HMG-CoA reductase inhibitors such as atorvastatin, cerivastatin, fluvastatin, lovastatin, pravastatin, rosuvastatin, simvastatin, abciximab, angiopeptin, colchicines, eptifibatide, hirudin, methotrexate, streptokinase, taxol, ticlopidine, tissue plasminogen activator, trapidil, urokinase, vascular endothelial growth factor, transforming growth factor beta, insulin growth factor, platelet-derived growth factor, fibroblast growth factor, combinations thereof, etc. Co-Solvents: chloroform, acetone, methylene chloride, ethyl acetate and tetrahydrofuran (THF). As will be understood by one of ordinary skill in the art and described further below, however, there are many other appropriate polymers, bioactive materials and cosolvents that can be used.

A more complete listing of polymers that can be used in accordance with the present invention include rapidly bioerodible polymers such as, without limitation, poly[lactide-co-glycolide], polyanhydrides, and polyorthoesters, whose carboxylic groups are exposed on the external surface as their smooth surface erodes. In addition, polymers containing labile bonds, such as, without limitation, polyanhydrides and polyesters can also be used. Representative natural polymers that can be used include, without limitation, proteins, such as zein, modified zein, casein, gelatin, gluten, serum albumin, or collagen, and polysaccharides, such as, without limitation, cellulose, dextrans, polyhyaluronic acid, polymers of acrylic and methacrylic esters and alginic acid. Representative synthetic polymers that can be used in accordance with the present invention include, without limitation, polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Synthetically modified natural polymers that can be used in accordance with the present invention include, without limitation, alkyl celluloses, hydroxyalkyl celluloses, cellulose ethers, cellulose esters, and nitrocelluloses. Other polymers that can be used in accordance with the present invention include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly(ethylene terephthalate), poly(vinyl acetate), polyvinyl chloride, polystyrene, polyvinyl pyrrolidone, and polyvinylphenol. Representative bioerodible polymers include polylactides, polyglycolides and copolymers thereof, poly(ethylene terephthalate), poly(butic acid), poly(valeric acid), poly(lactide-co-caprolactone), poly[lactide-co-glycolide], polyanhydrides, polyorthoesters, blends and copolymers thereof.

These described polymers can be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif. or else synthesized from monomers obtained from these suppliers using standard techniques.

In addition, a variety of bioactive materials can be appropriate for use in accordance with the present invention. Some of these bioactive materials include, without limitation, drugs such as altretamin, fluorouracil, amsacrin, hydroxycarbamide, asparaginase, ifosfamid, bleomycin, lomustin, busulfan, melphalan, chlorambucil, mercaptopurin, chlormethin, methotrexate, cisplatin, mitomycin, cyclophosphamide, procarbazin, cytarabin, teniposid, dacarbazin, thiotepa, dactinomycin, tioguanin, daunorubicin, treosulphan, doxorubicin, tiophosphamide, estramucin, vinblastine, etoglucide, vincristine, etoposid, vindesin, penicillin, ampicillin, nafcillin, amoxicillin, oxacillin, azlocillin, penicillin G, carbenicillin, penicillin V, dicloxacillin, phenethicillin, floxacillin, piperacillin, mecillinam, sulbenicillin, methicillin, ticarcillin, mezlocillin, cefaclor, cephalothin, cefadroxil, cephapirin, cefamandole, cephradine, cefatrizine, cefsulodine, cefazolin, ceftazidim, ceforanide, ceftriaxon, cefoxitin, cefuroxime, cephacetrile, latamoxef, cephalexin, amikacin, neomycin, dibekacyn, kanamycin, gentamycin, netilmycin, kanamycin, tobramycin, amphotericin B, novobiocin, bacitracin, nystatin, clindamycin, polymyxins, colistin, rovamycin, erythromycin, spectinomycin, lincomycin, vancomycin, chlortetracycline, oxytetracycline, demeclocycline, rolitetracycline, doxycycline, tetracycline, minocycline, chloramphenicol, rifamycin, rifampicin, thiamphenicol, sulfadiazine, sulfamethizol, sulfadimethoxin, sulfamethoxazole, sulfadimidin, sulfamethoxypyridazine, sulfafurazole, sulfaphenazol, sulfalene, sulfisomidin, sulfamerazine, sulfisoxazole, trimethoprim with sulfamethoxazole, sulfametrole, methanamine, norfloxacin, cinoxacin, nalidixic acid, nitrofurantoine, nifurtoinol, oxolinic acid; metronidazole; aminosalicyclic acid, isoniazide, cycloserine, rifampicine, ethambutol, tiocarlide, ethionamide, viomycin; amithiozone, rifampicine, clofazimine, sodium sulfoxone, diaminodiphenylsulfone, amphotericin B, ketoconazole, clotrimazole, miconazole, econazole, natamycin, flucytosine, nystatine, griseofulvin, aciclovir, idoxuridine, amantidine, methisazone, cytarabine, vidarabine, ganciclovir, chloroquine, iodoquinol, clioquinol, metronidazole, dehydroemetine, paromomycin, diloxanide, furoatetinidazole, emetine, chloroquine, pyrimethamine, hydroxychloroquine, quinine, mefloquine, sulfadoxine/pyrimethamine, pentamidine, sodium suramin, primaquine, trimethoprim, proguanil, antimony potassium tartrate, niridazole, antimony sodium dimercaptosuccinate, oxamniquine, bephenium, piperazine, dichlorophen, praziquantel, diethylcarbamazine, pyrantel parmoate, hycanthone, pyrivium pamoate, levamisole, stibophen, mebendazole, tetramisole, metrifonate, thiobendazole, niclosamide, acetylsalicyclic acid, mefenamic acid, aclofenac, naproxen, azopropanone, niflumic acid, benzydamine, oxyphenbutazone, diclofenac, piroxicam, fenoprofen, pirprofen, flurbiprofen, sodium salicyclate, ibuprofensulindac, indomethacin, tiaprofenic acid, ketoprofen, tolmetin, colchicine, allopurinol, alfentanil, methadone, bezitramide, morphine, buprenorfine, nicomorphine, butorfanol, pentazocine, codeine, pethidine, dextromoramide, piritranide, dextropropoxyphene, sufentanil, fentanyl, articaine, mepivacaine, bupivacaine, prilocaine, etidocaine, procaine, lidocaine, tetracaine, amantidine, diphenhydramine, apomorphine, ethopropazine, benztropine mesylate, lergotril, biperiden, levodopa, bromocriptine, lisuride, carbidopa, metixen, chlorphenoxamine, orphenadrine, cycrimine, procyclidine, dexetimide, trihexyphenidyl, baclofen, carisoprodol, chlormezanone, chlorzoxazone, cyclobenzaprine, dantrolene, diazepam, febarbamate, mefenoxalone, mephenesin, metoxalone, methocarbamol, tolperisone, levothyronine, liothyronine, carbimazole, methimazole, methylthiouracil and propylthiouracil and/or natural or synthetic hormones such as, without limitation, cortisol, deoxycorticosterone, flurohydrocortisone, beclomethasone, betamethasone, cortisone, dexamethasone, fluocinolone, fluocinonide, fluocortolone, fluorometholone, fluprednisolone, flurandrenolide, halcinonide, hydrocortisone, medrysone, methylprednisolone, paramethasone, prednisolone, prednisone, triamcinolone (acetonide), danazole, fluoxymesterone, mesterolone, dihydrotestosterone methyltestosterone, testosterone, dehydroepiandrosetone, dehydroepiandrostendione, calusterone, nandrolone, dromostanolone, oxandrolone, ethylestrenol, oxymetholone, methandriol, stanozolol methandrostenolone, testolactone, cyproterone acetate, diethylstilbestrol, estradiol, estriol, ethinylestradiol, mestranol, quinestrol chlorotrianisene, clomiphene, ethamoxytriphetol, nafoxidine, tamoxifen, allylestrenol, desogestrel, dimethisterone, dydrogesterone, ethinylestrenol, ethisterone, ethynadiol diacetate, etynodiol, hydroxyprogesterone, levonorgestrel, lynestrenol, medroxyprogesterone, megestrol acetate, norethindrone, norethisterone, norethynodrel, norgestrel, progesterone, inhibin, antidiuretic hormone, proopiomelanocortin, follicle stimulating hormone, prolactin, angiogenin, epidermal growth factor, calcitonin, erythropoietin, thyrotropic releasing hormone, insulin, growth hormones, human chorionic gonadotropin, luteinizing hormone, adrenocorticotropic hormone (ACTH), lutenizing hormone releasing hormone (LHRH), parathyroid hormone (PTH), thyrotropin releasing hormone (TRH), vasopressin, and corticotropin releasing hormone.

In certain embodiments, volatile solvents are those that have atmospheric boiling points below about 90° C., below about 80° C., below about 60° C. or below about 40° C. A more complete list of solvents that can be used in accordance with the present invention include, without limitation, chloroform, acetone, dimethylsulfoxide (DMSO), propylene glycol methyl ether (PM,) iso-propylalcohol (IPA), n-propylalcohol, methanol, ethanol, tetrahydrofuran (THF), dimethylformamide (DMF), dimethyl acetamide (DMAC), benzene, toluene, xylene, hexane, cyclohexane, heptane, octane, nonane, decane, decalin, ethyl acetate, butyl acetate, isobutyl acetate, isopropyl acetate, butanol, diacetone alcohol, benzyl alcohol, acetone, 2-butanone, cyclohexanone, dioxane, methylene chloride, carbon tetrachloride, tetrachlroro ethylene, tetrachloro ethane, chlorobenzene, 1,1,1-trichloroethane, formamide, and combinations thereof.

Thus, as should be evident from the preceding disclosure, a variety of polymers, bioactive materials, co-solvents, mold and evaporation systems and drying devices can be used in accordance with the present invention. Specific embodiments will include at a minimum forming an implantable medical device by dissolving a polymer and a bioactive material in a co-solvent to form a mixture; injecting the mixture into a mold that is part of a system comprising at least one evaporation port; allowing the co-solvent to evaporate from the mixture while the mixture is in the mold; and removing the mixture from the mold after the evaporation.

Other embodiments according to the present invention can modify or add steps or features to this basic embodiment by, without limitation: (i) accelerating the rate of co-solvent evaporation and/or by (ii) further drying or treating the mixture once it is removed from the mold. Accelerating the rate of co-solvent evaporation can be achieved by, without limitation, opening one or more evaporation ports; applying a vacuum to said one or more evaporation ports; heating the system to a temperature below that which would degrade the bioactive material; and combinations thereof. After removal from the mold, the mixture can be further dried by, without limitation, placing the mixture in at least one device selected from the group consisting of an oven, a vacuum oven, a vacuum chamber, a fume hood and a laminar flow hood. Further treatments can include, without limitation, adding additional drug layers or coatings to the surface of the created medical device.

The present invention provides methods to produce a variety of medical devices. In one embodiment, these methods are used to produce stents. The methods can be used to create a variety of other medical devices, however, including, without limitation, those described in the preceding provided definition of “medical devices.”

Unless otherwise indicated, all numbers expressing quantities of ingredients, properties such as molecular weight, reaction conditions, and so forth are to be understood as being modified in all instances by the term “about.” Accordingly, unless indicated to the contrary, the numerical parameters set forth are approximations that may vary depending upon the desired properties sought to be obtained. At the very least, each numerical parameter should at least be construed in light of the number of reported significant digits and by applying ordinary rounding techniques. Notwithstanding that the numerical ranges and parameters are approximations, the numerical values set forth in the specific examples are reported as precisely as possible. Any numerical value, however, inherently contains certain errors necessarily resulting from the standard deviation found in their respective testing measurements.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

While certain embodiments according to this invention are described herein, variations of those embodiments will become apparent to those of ordinary skill in the art upon reading the foregoing description.

Furthermore, numerous references have been made to patents and printed publications throughout this specification. Each of the above cited references and printed publications are herein individually incorporated by reference in their entirety.

What is claimed is:

1. A method of forming an implantable medical device comprising:
dissolving a polymer and a bioactive material in a co-solvent to form a mixture;
injecting said mixture into a mold;
allowing said co-solvent to evaporate from said mixture while said mixture is in said mold; and
removing said mixture from said mold after said evaporation.

2. A method according to claim 1 wherein said mold is part of a system comprising at least one evaporation port and the rate of evaporation of said co-solvent is accelerated through a method selected from the group consisting of opening one or more evaporation ports; applying a vacuum to said one or more evaporation ports; heating said system to a temperature below that which would degrade said bioactive material; and combinations thereof.

3. A method according to claim 1 wherein said method further comprises drying said mixture after removal from said mold.

4. A method according to claim 3 wherein said drying occurs through placing said mixture in at least one device selected from the group consisting of an oven, a vacuum oven, a vacuum chamber, a fume hood and a laminar flow hood.

5. A method according to claim 1 wherein said polymer is selected from the group consisting of polyesters, polyacrylamides, polyvinylpyrrolidone, polymethylmethacrylate, polybutylmethacrylate, polyvinyl acetate, poly-lactic acid (PLA), poly-glycolic acid (PGA), polycarbonates, polyurethanes, polycapralactone, polyorthoester and copolymers thereof.

6. A method according to claim 1 wherein said bioactive material is selected from the group consisting of Zotarolimus (ABT-578), rapamycin, paclitaxel, dexamethasone, everolimus, tacrolimus, des-aspartate angiotensin I, exochelins, nitric oxide, apocynin, gamma-tocopheryl, pleiotrophin, estradiol, heparin, aspirin, atorvastatin, cerivastatin, fluvastatin, lovastatin, pravastatin, rosuvastatin, simvastatin, abciximab, angiopeptin, colchicines, eptifibatide, hirudin, methotrexate, streptokinase, taxol, ticlopidine, tissue plasminogen activator, trapidil, urokinase, vascular endothelial growth factor, transforming growth factor beta, insulin growth factor, platelet-derived growth factor, fibroblast growth factor, and combinations thereof.

7. A method according to claim 1 wherein said co-solvent is selected from the group consisting of dimethylsulfoxide, iso-propyl alcohol, methanol, ethanol, dimethylformamide, benzene, toluene, xylene, cyclohexane, heptane, chloroform, acetone, methylene chloride, ethyl acetate, tetrahydrofuran (THF), and combinations thereof.

8. A method according to claim 1 wherein said implantable medical device is a stent.

9. A method according to claim 1 wherein said implantable medical device is a stent, said polymer is selected from the group consisting of polyesters, polyacrylamides, polyvinylpyrrolidone, polymethylmethacrylate, polybutylmethacrylate, polyvinyl acetate, poly-lactic acid (PLA), poly-glycolic acid (PGA), polycarbonates, polyurethanes, polycapralactone, polyorthoester and copolymers thereof, said bioactive material is selected from the group consisting of Zotarolimus (ABT-578), rapamycin, paclitaxel, dexamethasone, everolimus, tacrolimus, des-aspartate angiotensin I, exochelins, nitric oxide, apocynin, gamma-tocopheryl, pleiotrophin, estradiol, heparin, aspirin, atorvastatin, cerivastatin, fluvastatin, lovastatin, pravastatin, rosuvastatin, simvastatin, abciximab, angiopeptin, colchicines, eptifibatide, hirudin, methotrexate, streptokinase, taxol, ticlopidine, tissue plasminogen activator, trapidil, urokinase, vascular endothelial growth factor, transforming growth factor beta, insulin growth factor, platelet-derived growth factor, fibroblast growth factor, and combinations thereof and said co-solvent is selected from the group consisting of dimethylsulfoxide, iso-propyl alcohol, methanol, ethanol, dimethylformamide, benzene, toluene, xylene, cyclohexane, heptane, chloroform, acetone, methylene chloride, ethyl acetate, tetrahydrofuran (THF) and combinations thereof.

10. An implantable medical device wherein said medical device comprises a polymer and a bioactive material wherein at one point said polymer and bioactive material were dissolved together in a co-solvent and injected into a mold and wherein the temperature of said co-solvent was kept below a temperature at which said bioactive material would degrade.

11. An implantable medical device according to claim 10 wherein said co-solvent was allowed to evaporate from said mold.

12. An implantable medical device according to claim 11 wherein said mold was part of a system comprising at least one evaporation port and the rate of said evaporation was accelerated through a method selected from the group consisting of opening one or more evaporation ports; applying a vacuum to said one or more evaporation ports; heating said system to a temperature below that which would degrade said bioactive material; and combinations thereof.

13. An implantable medical device according to claim 11 wherein said polymer and bioactive material were removed from said mold after said evaporation of said co-solvent and further dried.

14. An implantable medical device according to claim 13 wherein said drying occurred in at least one device selected from the group consisting of an oven, a vacuum oven, a vacuum chamber, a fume hood and a laminar flow hood.

15. An implantable medical device according to claim 10 wherein said polymer is selected from the group consisting of polyesters, polyacrylamides, polyvinylpyrrolidone, polymethylmethacrylate, polybutylmethacrylate, polyvinyl acetate, poly-lactic acid (PLA), poly-glycolic acid (PGA), polycarbonates, polyurethanes, polycapralactone, polyorthoester and copolymers thereof.

16. An implantable medical device according to claim 10 wherein said bioactive material is selected from the group consisting of Zotarolimus (ABT-578), rapamycin, paclitaxel, dexamethasone, everolimus, tacrolimus, des-aspartate angiotensin I, exochelins, nitric oxide, apocynin, gamma-tocopheryl, pleiotrophin, estradiol, heparin, aspirin and HMG-CoA reductase inhibitors such as atorvastatin, cerivastatin, fluvastatin, lovastatin, pravastatin, rosuvastatin, simvastatin, abciximab, angiopeptin, colchicines, eptifibatide, hirudin, methotrexate, streptokinase, taxol, ticlopidine, tissue plasminogen activator, trapidil, urokinase, vascular endothelial growth factor, transforming growth factor beta, insulin growth factor, platelet-derived growth factor, fibroblast growth factor, and combinations thereof.

17. An implantable medical device according to claim 10 wherein said co-solvent is selected from the group consisting of dimethylsulfoxide, iso-propyl alcohol, methanol, ethanol, dimethylformamide, benzene, toluene, xylene, cyclohexane, heptane, chloroform, acetone, methylene chloride, ethyl acetate, tetrahydrofuran (THF) and combinations thereof.

18. An implantable medical device according to claim 10 wherein said implantable medical device is a stent.

19. An implantable medical device according to claim 10 wherein said implantable medical device is a stent, said polymer is selected from the group consisting of polyesters, polyacrylamides, polyvinylpyrrolidone, polymethylmethacrylate, polybutylmethacrylate, polyvinyl acetate, poly-lactic acid (PLA), poly-glycolic acid (PGA), polycarbonates, polyurethanes, polycapralactone, polyorthoester and copolymers thereof, said bioactive material is selected from the group consisting of Zotarolimus (ABT-578), rapamycin, paclitaxel, dexamethasone, everolimus, tacrolimus, des-aspartate angiotensin I, exochelins, nitric oxide, apocynin, gamma-tocopheryl, pleiotrophin, estradiol, heparin, aspirin and HMG-CoA reductase inhibitors such as atorvastatin, cerivastatin, fluvastatin, lovastatin, pravastatin, rosuvastatin, simvastatin, abciximab, angiopeptin, colchicines, eptifibatide, hirudin, methotrexate, streptokinase, taxol, ticlopidine, tissue plasminogen activator, trapidil, urokinase, vascular endothelial growth factor, transforming growth factor beta, insulin growth factor, platelet-derived growth factor, fibroblast growth factor, and combinations thereof and said co-solvent is selected from the group consisting of dimethylsulfoxide, iso-propyl alcohol, methanol, ethanol, dimethylformamide, benzene, toluene, xylene, cyclohexane, heptane, chloroform, acetone, methylene chloride, ethyl acetate, tetrahydrofuran (THF) and combinations thereof.
